(19) United States
(12) Patent Application Publication
(10) Pub. No.: US 2015/0085136 A1
(43) Pub. Date: Mar. 26, 2015

(54) Hybrid single-pixel camera switching mode for spatial and spot/area measurements

(71) Applicant: Xerox Corporation, Norwalk, CT (US)

(72) Inventors: Edgar A. Bernal, Webster, NY (US); Lalit Keshav Mestha, Fairport, NY (US); Beilei Xu, Penfield, NY (US)

(73) Assignee: Xerox Corporation, Norwalk, CT (US)

(21) Appl. No.: 14/037,847

(22) Filed: Sep. 26, 2013

(51) Int. Cl. H04N 5/30; H04N 5/33

(52) U.S. Cl. CPC H04N 5/30; H04N 5/33
USPC 348/164; 348/335

(57) ABSTRACT

Disclosed herein is a single-pixel camera system and method for performing spot/area measurement of a localized area of interest identified in a scene and for performing spatial scene reconstruction. A switching module enables a single-pixel camera to alternate between a spot/area measurement mode and a spatial scene reconstruction mode. In the case where the operative mode is switched to spot measurement, a light modulation device is configured to modulate incoming light according to a clustered pattern that is specific to a localized area of interest intended to be measured by integrating across the pixels to generate an integral value. In the case where the operative mode is switched to spatial scene reconstruction, the light modulation device can be configured to modulate incoming light to display a spatial pattern corresponding to a set of predetermined basis functions.

### BACKGROUND

The traditional single-pixel camera architecture computes random linear measurements of a scene under view and reconstructs the image of the scene from the measurements. The linear measurements are inner products between an N-pixel sampled version of the incident light field from the scene and a set of two-dimensional basis functions. The pixel-wise product is implemented via a digital micromirror device (DMD) consisting of a two-dimensional array of N mirrors that reflect the light towards a single photodetector or away from it. The photodetector integrates the pixel-wise product, which is an estimate of an inner (dot) product, and converts it to an output voltage. Spatial reconstruction of the image is possible by judicious processing of the set of estimated inner product values. There are scenarios where integration of reflectance scene values across specific spatial locations and/or temporal intervals, rather than, or in addition to spatial reconstruction of a scene, is desired.

### BRIEF DESCRIPTION

The present disclosure proposes a modification of the traditional single-pixel camera architecture to enable spot measurement in addition to spatial reconstruction of a scene. The system comprises the following modules: (1) a single-pixel-camera-based spatial scene reconstruction module; (2) a region of interest localization module; and, (3) a single-pixel-camera-based spot measurement module. A unique single-pixel camera with two different switching modes is used to enable modules 1 and 3 above. In the case of module 1, the DMD is configured to display basis functions that enable spatial scene reconstruction. In the case of module 3, the DMD displays clustered binary patterns having ON pixels at the locations indicated by module 2.

The present disclosure provides a method for using a single-pixel camera system for spot measurement. The method comprises: configuring a light modulation device comprising an array of imaging elements to spatially modulate incoming light according to a clustered pattern that enables spot measurement of a localized area of interest, the clustered pattern being specific to the localized area; and, measuring, using a photodetector of a single-pixel camera, a magnitude of an intensity of the modulated light across pixel locations in the clustered pattern. The magnitude of an intensity being equivalent to an integral value of the scene across the pixel locations, wherein the integral value comprises a spot measurement.

The present disclosure further provides a method for using a single-pixel camera system for spot measurement and spatial scene reconstruction. The method comprises: in response to a light modulation device comprising an array of imaging elements being configured to modulate incoming light according to a clustered pattern that enables spot measurement of a localized area of interest, wherein the clustered pattern can be specific to the localized area: measuring, using a photodetector of a single-pixel, a magnitude of an intensity of the modulated light across pixel locations in said clustered pattern; this operation being equivalent to integrating across the pixels, the integral value comprising a spot measurement; and, in response to the light modulation device being configured to modulate incoming light according to a multiplicity of spatial patterns that enable spatial scene reconstruction: measuring, using the photodetector, a magnitude of multiple intensities corresponding to the light being modulated by the different spatial patterns; and, reconstructing a spatial appearance of a scene from the measurements to obtain a spatially reconstructed scene.

The present disclosure also further provides a method for using a single-pixel camera system for spot measurement of a localized area of interest identified in a spatially reconstructed scene, the method comprising: processing a spatially reconstructed scene to identify pixels associated with a localized area of interest in the scene as being active, with pixels outside the localized area being inactive pixels; configuring a light modulation device comprising an array of imaging elements to modulate incoming light according to a spatial pattern corresponding to the active pixels; measuring, using a photodetector of a single-pixel camera, a magnitude of an intensity of the modulated light across the active pixels; the measurement being equivalent to integrating across the active pixels to generate an integral value thereof, the integral value comprising a spot measurement of the localized area.

The present disclosure yet further provides a single-pixel camera system for performing spot measurement and spatial scene reconstruction, the camera system comprising: a light modulation device comprising a configurable array of imaging elements which modulate incoming light of a scene; a switch for toggling a configuration of the light modulation device to a first state wherein the array of imaging elements are configured according to a clustered pattern which enables spot measurement of a localized area of interest, and to a second state wherein the array of imaging elements are configured according to a multiplicity of spatial patterns which enable spatial scene reconstruction; a photodetector for measuring an intensity of the modulated light, this measuring being equivalent to integrating; and, a processor receiving the measurements, wherein in response to the light modulation device being configured to the first state, said measurements comprise a spot measurement of the localized area of interest, and in response to the light modulation device being configured to the second state, said processor spatially reconstructing the scene from multiple measurements obtained by integrating the incoming light modulated by the multiplicity of spatial patterns.

### BRIEF DESCRIPTION OF THE DRAWINGS

FIG. 1 is chart illustrating the electromagnetic spectrum and prices of cameras sensitive to different portions of the spectrum.

FIG. 2 shows a sample implementation of a single-pixel camera prototype.

FIG. 3(a) is a schematic illustration of a unique single-pixel camera with two different switching modes.

FIG. 3(b) is a schematic illustration of an alternative embodiment of the embodiment shown in FIG. 3(a).

FIG. 4 illustrates temporal sequence of images of a body part where monochromatic video data of a subject's hand is used.

FIG. 5 illustrates the resulting binary mask from region of interest localization superimposed on the original reconstructed image from FIG. 3 wherein ON pixels are displayed in white, and OFF pixels are displayed in black.

FIG. 6(a) is a pseudo-random and optimized sample DMD pattern used by the single-pixel camera for spatial scene reconstruction.

FIG. 6(b) is a clustered sample DMD pattern used by the single-pixel camera for integral spot/area measurement.

### DETAILED DESCRIPTION

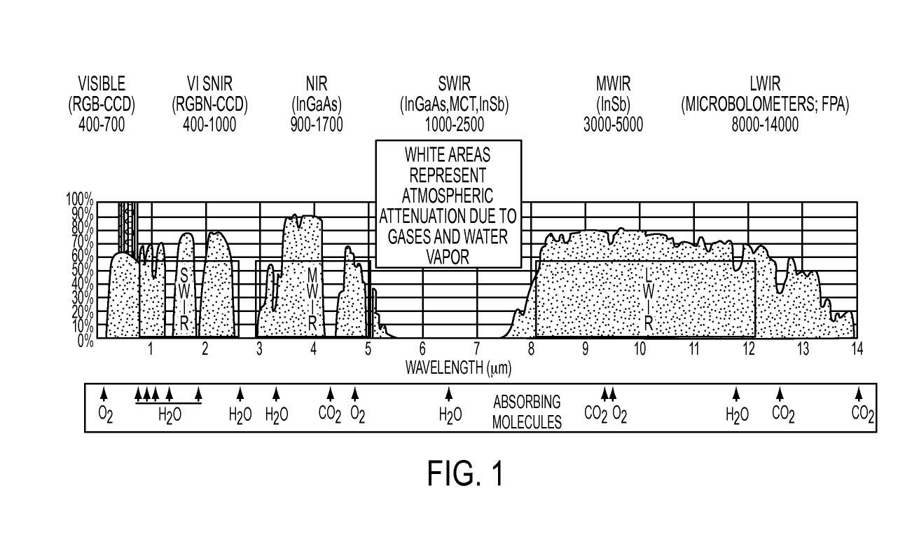

Consumer digital cameras in the megapixel range are commonplace due to the fact that silicon, the semiconductor material of choice for large-scale electronics integration, readily converts photons at visual wavelengths into electrons. On the other hand, imaging outside the visible wavelength range is considerably more expensive. FIG. 1 includes sample camera prices for different portions of the electromagnetic spectrum.

Hyperspectral and multispectral imaging has a wide range of applications. The most notable examples include medical/healthcare imaging (e.g., human vitals monitoring) and transportation (e.g., occupancy detection and remote vehicular emissions monitoring). It is thus desirable to find a less expensive alternative to traditional multispectral imaging solutions. The single-pixel camera design reduces the required cost of sensing an image by using one detector with extended sensitivity (e.g., infrared or ultraviolet) rather than a two-dimensional array of detectors with this expensive extended capability. The potential applications are significantly enhanced by using more than one wavelength band.

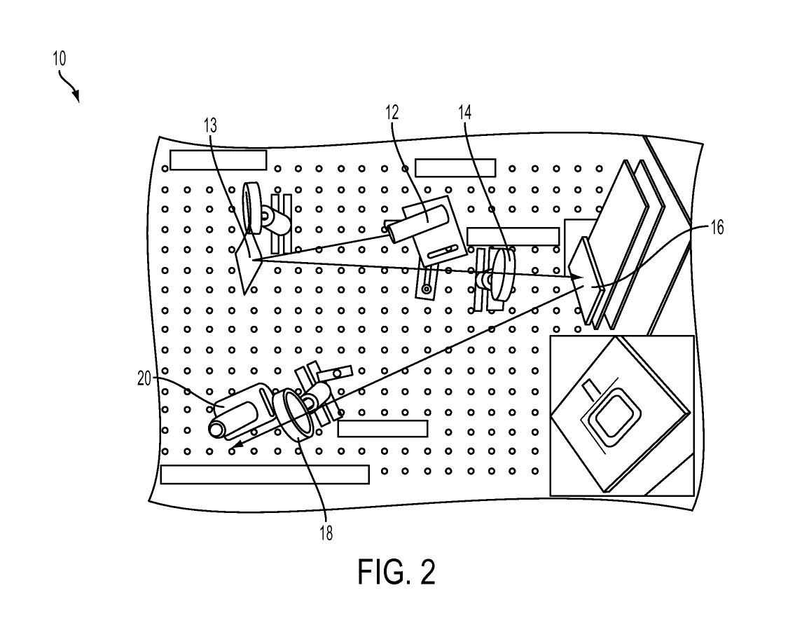

FIG. 2 shows a picture of components of a single-pixel camera 10. The camera comprises the following modules: a light source 12 which illuminates an object/scene 13 to be captured; an imaging lens 14 which focuses an image of the object 12 onto the DMD 16; the DMD 16 which performs pixel-wise inner product multiplication between incoming light and a set of predetermined basis functions; a collector lens 18 which focuses the light reflected from the DMD 16 inner product multiplication onto photodetector 20; the photodetector 20 which integrates or measures a magnitude of the inner product in the form of light intensity and converts it to voltage; and, a processing unit (not shown) which reconstructs the scene from inner product measurements as the various basis functions are applied over time.

If x[•] denotes the N-pixel sampled version of the image scene and φₘ[•] the m-th basis function displayed by the DMD 16, then each measurement performed by the photodetector 20 corresponds to an inner product yₘ=(x,φₘ). The mirror orientations corresponding to the different basis functions can typically be chosen using pseudorandom number generators (e.g., iid Gaussian, iid Bernoulli, etc.) that produce patterns with close to 50% fill factor. In other words, at any given time, about half of the micromirrors in the DMD 16 array are oriented towards the photodetector 20 while the complementary fraction is oriented away from it. By making the basis functions pseudorandom, the N-pixel sampled scene image x[•] can typically be reconstructed with significantly fewer samples than those dictated by the Nyquist sampling theorem (i.e., the image can be reconstructed after M inner products, where M<<N). Note that, N is the total number of mirrors in the DMD 16.

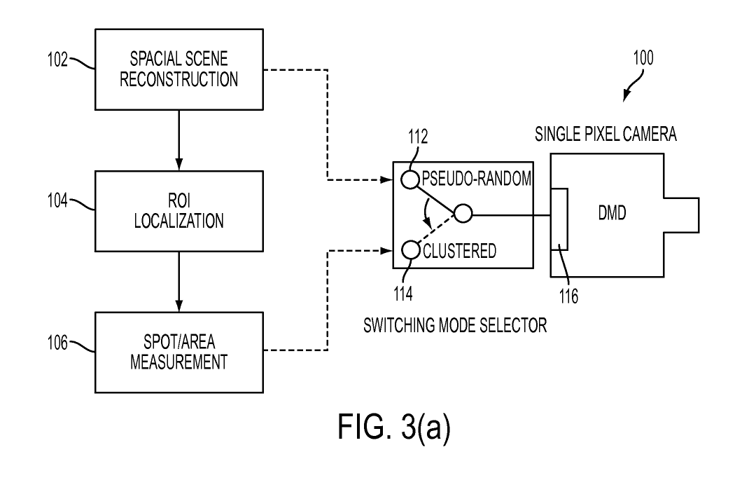

Referring to FIG. 3(a), the present disclosure provides for a proposes a modification of the traditional single-pixel camera 100 architecture which would enable spot measurement capabilities in addition to the conventional spatial reconstruction features. The system comprises the following modules: a single-pixel-camera-based spatial scene reconstruction module 102; a region of interest (ROI) localization module 104, and, a single-pixel-camera-based integral spot/area measurement module 106. It is to be appreciated that the light modulation switching device can comprise any of the following: a digital micromirror device (DMD), a transmissive liquid crystal modulator (LC), and a reflective liquid crystal on silicon (LCOS).

As FIG. 3(a) illustrates, the unique single-pixel camera 100 with two different switching modes 112, 114 can be used to enable modules 102 and 106. In the case of module 102, the DMD 116 is configured to display 112 basis functions that enable spatial scene reconstruction; in the case of module 106 the DMD 116 displays clustered 114 binary patterns having ON pixels at the locations indicated by module 104.

In an alternative embodiment such as the one illustrated in FIG. 3(b), the switching mode selector can additionally toggle between multiple spectral bands in a multi-band capable single-pixel camera. The embodiment illustrated in FIG. 3(b) could require reconstruction or spot/area measurement on a single band or a subset of available bands only at a given time. The switch can be toggled in response to any of the following: a manual input, acquisition of a predetermined number of spatial reconstruction data samples, a predetermined time interval, and an external event having occurred within the region or localized area of interest wherein the spot measurement is being performed.

The modules of the disclosure will be described hereinafter in the context of the application of heart rate estimation from localized vascular pathways. A vascular pathway localization application can be used to motivate the need for integral spot/area measurements of spatially reconstructed images. It will become apparent, however, that the proposed hybrid operating mode to be described hereinafter has a much broader applicability.

It has been demonstrated that it is possible to accurately estimate heart rate via non-contact methods based on analysis of video data of a person acquired with a traditional red-green-blue (RGB) camera. While demonstrations showed that robust heart rate estimation is possible from analysis of RGB data of the facial area of the subject, additional experiments showed that extending those techniques to other body parts including hands was problematic. Improvements in the accuracy of heart rate estimation can be achieved by analyzing integrated RGB data of pixels along the vascular pathway region of interest only, (i.e., vascular pathway localization module). Since hemoglobin has a higher absorption rate in the near infra-red (NIR) band than other tissues, the localization module processed data captured with an NIR imaging device. Once the pixel coordinates corresponding to vascular pathway regions of interest (ROI) were identified, integration of the RGB signals across the detected ROI pixel locations improved heart rate estimation, to the point where the technique was successfully applied on images of hands. Robust vascular pathway localization is also possible in the visible domain, by analyzing RGB signals both spatially and in time. Thus, an NIR imaging device for localization is no longer needed. Further improvements over the heart rate estimation results can be achieved with this technique by first locating pixels corresponding to the vascular pathway and then spatially and temporally integrating RGB data across the located ROI pixels.

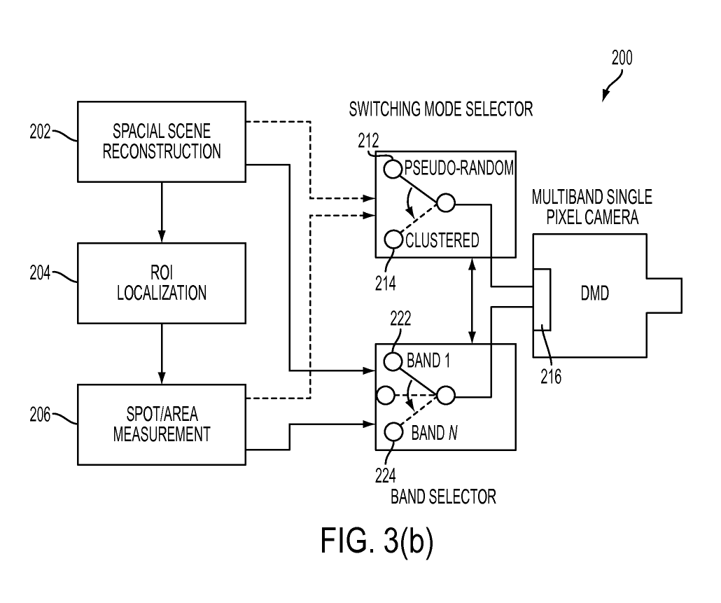

Other applications are related to glucose or bilirubin measurements with spot reflectance data at multiple wavelengths, the capability offered by the dual-beam single-pixel camera architecture. A spatial scene reconstruction module can be composed of a traditional single-pixel camera as described above and as illustrated in FIG. 2. The output of this module is an image or a temporal sequence of images of a body part of the subject of interest, as illustrated in FIG. 4 where monochromatic video data of the subject's hand 300 is used. Note that while the traditional single-pixel architecture enables single band capture only (hence the monochromatic video 300), extensions of the architecture to multi-band capabilities can be used to perform the spatial reconstruction. In a heart rate estimation application, the spatial scene reconstruction module can use an NIR-capable photodetector given the higher contrast of hemoglobin in the NIR, although an RGB-capable single-pixel camera can also be used.

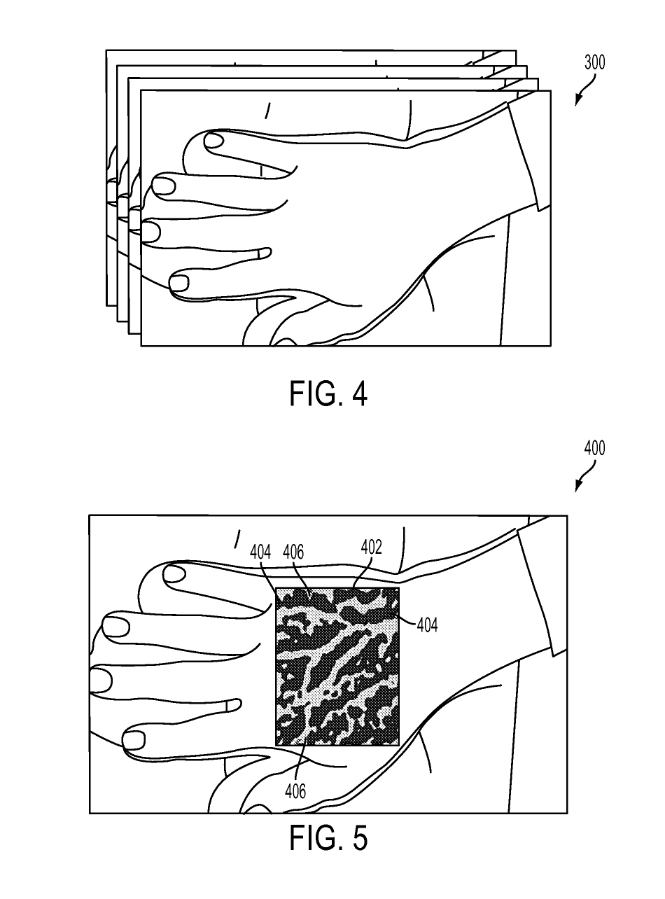

A localized region or area of interest 402 (i.e., image segment) can apply vascular pathway localization techniques to the spatially reconstructed image or video 400. When the spatial reconstruction is performed in the NIR band, the localization technique can be applied. The output of this module is a binary mask having pixel values equal to 1 at image locations where the vascular pathway is present 404, and having pixel values equal to 0 elsewhere 406, as illustrated in FIG. 5. It is to be appreciated that the mask generation module can process the scene and identify active pixels associated with the localized area of interest, wherein the pixels outside the localized area being identified as inactive pixels, the clustered pattern corresponding to the active pixels, and the integration occurring from measurements across all pixels identified by the mask as being active. The mask can further be generated from the spatially reconstructed scene. The mask can be automatically determined using any of the following: object identification, pixel classification, material analysis, texture identification, a facial recognition, and a pattern recognition method. Alternatively, a mask created by an operator via manual localization of a region of interest can be used.

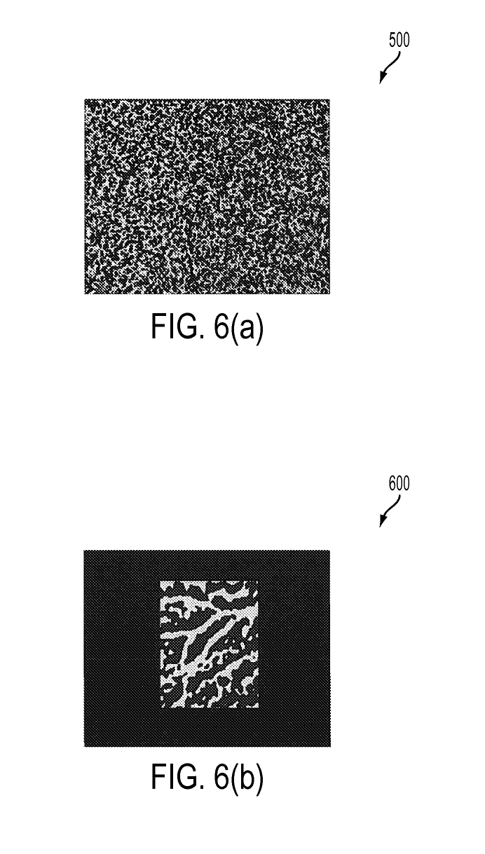

Robust heart rate estimation relies on the integration of RGB values across the localized ROI module. This integration can be done as a post-processing stage on the localized ROI 402. A single-pixel camera enables seamless integration by configuring the DMD to display clustered patterns corresponding to the detected ROI. FIG. 6 shows two sample DMD patterns: the pattern 500 from FIG. 6(a) is pseudo-random and optimized for spatial reconstruction, while the pattern 600 from FIG. 6(b) is clustered and optimal for integral spot/area measurements. The pattern used for spatial reconstruction can correspond to any of the following: one dimensional orthonormal, two dimensional orthonormal, one dimensional pseudo-random, two dimensional pseudo-random, one dimensional clustered, two dimensional clustered, natural, Fourier, wavelength, noise length, and discrete co-sign transform (DCT) as basis functions. The region for localized area of interest can be determined by using any of the following: object identification, pixel classification, material analysis, texture identification, facial recognition, and pattern recognition methods.

In one embodiment, the switch from spatial scene reconstruction to spot measurement mode occurs after the full scene reconstruction has been achieved. Typically, this is achieved after M sparse measurements of the scene, where M is in the order of K log(1+N/K). Here, K is the sparsity order of the scene and N is the number of pixels in the DMD. It is to be appreciated that integration of spot measurement may also be performed over time to increase the number of photons (i.e., signal to noise ratio). As such, different integration time lengths can be assigned to different regions of interest in the mask, thus effectively achieving a relative weighting of the different spot measurements. This is denoted integration weighting. One way of performing integration weighting can be achieved by utilizing masks with a number of levels that is greater than 2. For example, a mask can contain two different regions of interest, region one associated with mask values equal to 0.5 and region two associated with values equal to 1. The mask value associated with a given region of interest can be indicative (e.g., directly proportional) of the integration time associated with said region of interest. In this case, the total integration time associated with region one may be half the integration time associated with region two. Different integration times for different regions of interest can be achieved, for example, by pulse modulation weighting, whereby judicious pulse modulation of the micromirrors associated with the different regions of interest is implemented, in which case the duty cycle of the signal controlling the ON and OFF positions of a micromirror in region one may be half the duty cycle of the signal controlling the ON and OFF positions of a micromirror in region two. Alternatively, a sequential weighting scheme can be implemented whereby micromirrors associated with both regions are in the ON position for half the exposure cycle, and then the micromirrors associated with region one are turned OFF for the remaining of the exposure cycle. According to yet another type of weighting denoted spatial weighting, half of the micromirrors associated with region one are configured to the ON position for the full exposure cycle, while the full set of micromirrors associated with region two are configured to the ON position for the full exposure cycle. Combinations of the different weighting strategies are also possible. Note that other, non-linear relationships between mask values and integration times can be utilized.

In another embodiment, full spatial reconstruction is not a prerequisite for the switch to occur. For example, in one case, it may be enough to have edge information of the scene before performing the switch. This typically can be achieved sooner (with fewer measurements) than the full spatial reconstruction of the scene.

The original vision for single-pixel camera devices was aimed at sparse spatial reconstruction of scenes. The present disclosure teaches away from originally taught embodiments by proposing an alternative switching mode that does not rely on sparsity alone. Processing of measurements is performed in-camera rather than offline, and switching is done based on the scene/requirements for hybrid mode capture. Since fill factor and photometric efficiency of single photodetectors are larger than those provided by single sensors in 2D arrays, integral measurements are expected to be more robust to noise than those performed offline on images.

Additional scenarios where the proposed architecture can be useful include:

a) Non-invasive glucose/blood/tissue content detection, in which multiple spectral bands are captured on a single spot on the human body (e.g. back of the palm, face, chest etc.). Here, the single-pixel camera will act as a spot spectral measurement device. Blood content detection includes the measurement of bilirubin, glucose and other compounds such as creatinine, urea, melanin etc.;

b) Non-contact patient temperature monitoring via the use of a photodetector sensitive in the mid to long IR band; and, c) Occupancy detection, where the spatial reconstruction will be used to locate the potential passenger location and the spot measurement will be used to verify the presence of skin in order to establish whether the detected object corresponds to a human or a dummy.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

What is claimed is:

1. A method for using a single-pixel camera system for spot measurement, the method comprising:
configuring a light modulation device comprising an array of imaging elements to spatially modulate incoming light according to a clustered pattern that enables spot measurement of a localized area of interest, said clustered pattern being specific to said localized area;
measuring, using a photodetector of a single-pixel camera, a magnitude of an intensity of said modulated light across pixel locations in said clustered pattern;
wherein said magnitude of an intensity is equivalent to an integral value of the scene across said pixel locations; and,
wherein said integral value comprises a spot measurement.

2. The method of claim 1, wherein said light modulation device is selected from the group consisting of: a digital micromirror device (DMD), a transmissive liquid crystal modulator (LC), and a reflective liquid crystal on silicon (LCOS).

3. The method of claim 1, wherein said photodetector is capable of detecting any of: an infrared wavelength band, an ultraviolet band, and a visible wavelength band.

4. The method of claim 1, wherein said integration is performed at a specified time.

5. The method of claim 1, further comprising receiving a mask which identifies pixels associated with said localized area of interest in a scene as being active, with pixels outside said localized area being inactive, said clustered pattern corresponding to active pixels identified by said mask, said photodetector measuring a magnitude of an intensity of said modulated light across said active pixels, and said integration occurring across said active pixels.

6. The method of claim 5, wherein said integration is weighted.

7. A method for using a single-pixel camera system for spot measurement and spatial scene reconstruction, the method comprising:
in response to a light modulation device comprising an array of imaging elements being configured to modulate incoming light according to a clustered pattern that enables spot measurement of a localized area of interest, said clustered pattern being specific to said localized area:
measuring, using a photodetector of a single-pixel camera, a magnitude of an intensity of said modulated light across pixel locations in said clustered pattern, said magnitude corresponding to an integral value across said pixels, said integral value comprising a spot measurement; and,
in response to said light modulation device being configured to modulate incoming light according to a multiplicity of spatial patterns that enable spatial scene reconstruction:
measuring, using said photodetector, a magnitude of multiple intensities corresponding to the light being modulated by different spatial patterns; and,
reconstructing a spatial appearance of a scene from said measurements to obtain a spatially reconstructed scene.

8. The method of claim 7, wherein said light modulation device comprises any of: a digital micromirror device (DMD), a transmissive liquid crystal modulator (LC), and a reflective liquid crystal on silicon (LCOS).

9. The method of claim 7, wherein said photodetector is capable of detecting any of: an infrared wavelength band, an ultraviolet band, and a visible wavelength band.

10. The method of claim 7, wherein said integration is performed at a specified time.

11. The method of claim 7, wherein said spatial pattern corresponds to any of: 1D orthonormal, 2D orthonormal, 1D pseudorandom, 2D pseudorandom, 1D clustered, 2D clustered, natural, Fourier, wavelet, noiselet, and Discrete Cosine Transform (DCT) basis functions.

12. The method of claim 7, wherein said integration is weighted.

13. A method for using a single-pixel camera system for spot measurement of a localized area of interest identified in a spatially reconstructed scene, the method comprising:
processing a spatially reconstructed scene to identify pixels associated with a localized area of interest in said scene as being active, with pixels outside said localized area being inactive pixels;
configuring a light modulation device comprising an array of imaging elements to modulate incoming light according to a spatial pattern corresponding to said active pixels;
measuring, using a photodetector of a single-pixel camera, a magnitude of an intensity of said modulated light across said active pixels, said measurement being equivalent to integrating across said active pixels to generate an integral value thereof, said integral value comprising a spot measurement of said localized area.

14. The method of claim 13, wherein said light modulation device comprises any of: a digital micromirror device (DMD), a transmissive liquid crystal modulator (LC), and a reflective liquid crystal on silicon (LCOS).

15. The method of claim 13, wherein said integration is performed at a specified time.

16. The method of claim 13, wherein the location of said region of interest is identified by a mask, and said integration is weighted by the values of said mask.

17. The method of claim 13, wherein said localized area of interest is determined in using any of: object identification, pixel classification, material analysis, texture identification, a facial recognition, and pattern recognition methods; and,
a processor receiving the measurements, wherein in response to the light modulation device being configured to the first state said measurements comprise a spot measurement of the localized area of interest, and in response to the light modulation device being configured to the second state, said processor spatially reconstructing the scene from measurements obtained across all pixels identified by the multiplicity of spatial patterns.

18. A single-pixel camera system for performing spot measurement and spatial scene reconstruction, the camera system comprising:
a light modulation device comprising a configurable array of imaging elements which modulate incoming light of a scene;
a switch for toggling a configuration of said light modulation device to a first state wherein said array of imaging elements are configured according to a clustered pattern which enables spot measurement of a localized area of interest, and to a second state wherein said array of imaging elements are configured according to a multiplicity of spatial patterns which enable spatial scene reconstruction;
a photodetector for measuring an intensity of said modulated light, this measuring being equivalent to integrating; and,
a processor receiving said measurements, wherein in response to said light modulation device being configured to said first state, said measurements comprise a spot measurement of the localized area of interest, and in response to said light modulation device being configured to said second state, said processor spatially reconstructing said scene from multiple measurements obtained by integrating the incoming light modulated by said multiplicity of spatial patterns.

19. The camera system of claim 18, wherein said light modulation device comprises any of: a digital micromirror device (DMD), a transmissive liquid crystal modulator (LC), and a reflective liquid crystal on silicon (LCOS).

20. The camera system of claim 18, wherein said switch is toggled in response to any of: a manual input, acquisition of a predetermined number of spatial reconstruction data samples, a predetermined time interval, and an external event having occurred within said localized area of interest wherein said spot measurement is being performed.

21. The camera system of claim 18, further comprising a mask generation module which processes said scene and identifies active pixels associated with said localized area of interest, with pixels outside said localized area being identified as inactive pixels, said clustered pattern corresponding to said active pixels, and said integration occurring from measurements across all pixels identified by said mask as being active.

22. The camera system of claim 21, wherein said mask is generated from said spatially reconstructed scene.

23. The camera system of claim 21, wherein said mask is determined using any of: object identification, pixel classification, material analysis, texture identification, a facial recognition, and a pattern recognition method.
